(19) United States
(12) Patent Application Publication
(10) Pub. No.: US 2022/0031664 A1
(43) Pub. Date: Feb. 3, 2022

(54) PHARMACEUTICAL COMPOSITIONS COMPRISING ANTIEMETICS AND ALPHA-2-ADRENERGIC RECEPTOR AGONISTS AND METHODS OF USING THE SAME FOR ANESTHESIOLOGICAL APPLICATIONS

(71) Applicant: Melt Pharmaceuticals, Inc., San Diego, CA (US)

(72) Inventor: Dennis Elias Saadeh, Brentwood, TN (US)

(21) Appl. No.: 16/940,740

(22) Filed: Jul. 28, 2020

(51) Int. Cl. A61K 31/4178; A61K 31/4164; A61K 31/138

(52) U.S. Cl. CPC A61K 31/4178; A61K 31/138; A61K 31/4164

(57) ABSTRACT

Pharmaceutical compositions and methods for inducing conscious sedation using such compositions are described, the compositions including antiemetic(s) and a-2-adrenergic receptor agonist(s), and optionally β-blocker(s), NSAID(s), antihistamine(s), and/or pain reliever(s). The compositions may be incorporated into vehicles for extended release. Methods for fabricating the compositions and using them for anesthesiological applications are also described.

### FIELD OF THE INVENTION

The present invention relates generally to the field of pharmacology and more specifically to compositions having anesthetic properties that are useful in various kinds of medical practice, such as surgery, and to methods of preparing and using such compositions.

### BACKGROUND

The present disclosure relates to solid or liquid pharmaceutical formulations comprising combinations of active agents such as anti-emetic(s), α-2-adrenergic receptor agonist(s), and optionally additional pharmaceutically active compound(s) such as β-blocker(s), NSAID(s), antihistamine(s), and/or pain reliever(s) as well as methods for using the same for providing anesthesia by administering such compositions orally or otherwise, including such administrations as sublingual or buccal.

It is necessary in many cases to use local anesthesia, particularly via oral route in the course of various surgical procedures, e.g., ophthalmic surgeries or urological interventions. For instance, when local anesthesia is employed during or prior to intraocular operations, the occurrences of pain, anxiety, peri-operative stress, nausea, agitation, vomiting and the like are less frequent, which will typically have a very beneficial effect on the surgical experience and reduce the number of intraocular complications such as bleeding, secretions, cardiac and/or pulmonary complications, etc. The severity of those complications when they do occur will also be less pronounced when local anesthesia is used.

Traditionally intravenous route is used to administer medications for inducing general anesthesia. Alternatives to intravenous methods and therapies have been suggested and previously used for inducing general anesthesia. In particular, oral administration of benzodiazepines, opioid analgesics, propofol, ketamine or etomidate utilizing the MAC procedure (monitored anesthesia care) has been suggested and tried, but no more than minimal to moderate improvement has been achieved by such methods. Therefore, there remains a need for better formulations to achieve anesthesia in a patient.

This patent specification discloses such pharmaceutical compositions suitable for anesthesiological applications that can achieve positive patient outcomes while being free of the drawbacks and deficiencies of existing methods and formulations. Methods of fabricating and administering the same are also discussed.

### SUMMARY

According to various embodiments of the invention, there are provided pharmaceutical compositions. The compositions include a therapeutically effective quantity of at least one antiemetic medicament and at least one α-2-adrenergic receptor agonist.

In various embodiments of the invention, the pharmaceutical compositions described above may further optionally include a therapeutically effective quantity of at least one additional pharmaceutically active compound such as a β-blocker, an antihistamine, a nonsteroidal anti-inflammatory drug (NSAID), a pain reliever, or a combination thereof, or pharmaceutically acceptable salts, hydrates, solvates or N-oxides thereof.

In various embodiments of the invention, the pharmaceutical compositions described above may also optionally include a pharmaceutically acceptable excipient, which in some embodiments may provide for extended release of the pharmaceutical formulation.

According to further embodiments of the invention, the invention provides methods of using the composition(s) for inducing conscious sedation or pre-sedation in a subject. The methods include administering to a patient in need thereof (i.e., those patients who require conscious sedation or pre-sedation) a pharmaceutical composition described herein as the first step of a medical or surgical procedure, the procedure being an ophthalmic surgery (e.g., cataract surgery, glaucoma surgery, corneal surgery, eyelid surgery, or retinal surgery), a dental procedure (e.g., a tooth extraction, an oral surgery, or a root canal surgery), an outpatient medical procedure (e.g., medical imaging procedure, biopsy, bone marrow harvesting, colonoscopy, or endoscopy), a urological procedure, a laparoscopic procedure, obstetric and gynecological procedures, a gastrointestinal procedure, an otolaryngologic procedure, a cosmetic surgery procedure, a dermatological procedure, a podiatric procedure, an orthopedic procedure, an emergency medical treatment, a psychiatric treatment, or a veterinarian procedure.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20, as well as to the numbers in between integers, e.g., 1.5 or 2.5, and the like.

The term “pharmaceutical composition” is defined as a chemical or biological compound or substance, or a mixture or combination of two or more such compounds or substances, intended for use in medical diagnosis, cure, treatment, or prevention of disease or pathology.

The terms “anesthetic,” “anesthesia,” “anesthesiology” and the like refer herein to substances, compounds, processes or procedures that induce insensitivity to pain such as a temporary loss of sensation.

The term “conscious sedation” that for the purposes of this application may be used interchangeably with terms “procedural sedation” and “analgesia” is used herein to refer to an induced state of sedation characterized by a minimally depressed consciousness such that the patient is able to continuously and independently maintain a patent airway, retain protective reflexes, and remain responsive to verbal cues and/or tactile or physical stimulation.

Conscious sedation is typically performed/induced to decrease the level of anxiety in a patient and to elicit an improved degree of cooperation from the patient prior to or during a procedure. Conscious sedation, therefore, refers to a condition that is medically different and distinct from deep sedation, which is the next level of sedation defined as depression of consciousness when the patient's ability to independently maintain ventilatory function may be impaired and he or she cannot be easily aroused; however, the patient will still purposefully respond to repeated or painful stimulation.

Conscious sedation is also clearly distinguishable for the purposes of the present application from the lower level of sedation (i.e., minimal sedation when the patient is able to maintain a normal response to verbal stimuli) as well as the highest level of sedation (i.e., general anesthesia when there is no response from the patient even with painful stimulus).

The term “pre-sedation” is defined for the purposes of this application as conscious sedation that is induced some time before a procedure, e.g., between 5 minutes and 1 hour prior.

The terms “solvate” and “hydrate” are used herein to indicate that a compound or substance is physically or chemically associated with a solvent for “solvates” such as water (for “hydrates”).

The term “antiemetic” is defined as a drug or medicament that treats, reduces, and/or prevents nausea and/or vomiting.

The term “α-2-adrenergic receptor agonist” is defined as a class of sympathomimetic agents that selectively stimulates alpha adrenergic receptors that are associated with sympatholytic properties; α-2-adrenergic receptor agonists are known to inhibit activity of the enzyme adenylyl cyclase.

The term “β-blocker” refers to a compound of any kind that can prevent or reduce the stimulation of the adrenergic receptors responsible for increased cardiac action.

The term “non-steroid anti-inflammatory drug” or “NSAID” refers to a class of compounds that are free of any steroid moieties yet are capable of providing analgesic, antipyretic and/or anti-inflammatory effects.

The term “antihistamine medicament” refers to any compound that is capable of inhibiting or counteracting the physiological effects of histamine.

The term “polyglycol” is defined as a polymer or oligomer containing several ether-glycol linkages that yields one or more glycols when these linkages are cleaved, e.g., by hydrolysis.

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, diluents and products used for facilitating drug absorption or solubility or for other pharmacokinetic considerations.

The term “binder” refers to a substance or compound that promotes, provides or improves cohesion, i.e., a substance that causes the components of a mixture to cohere to form a solid item that possesses integrity.

The term “troche” refers to a small tablet or lozenge (i.e., a medicated candy intended to be dissolved in the mouth), typically in a form of a disk, a ball or rhombic in cross-section, comprising medication that is processed into a paste and dried.

The term “therapeutically effective amount” is defined as the amount of a compound or pharmaceutical composition that will elicit the biological or medical response of a tissue, system, animal or human that is being sought by the researcher, medical doctor or other clinician.

The term “pharmaceutically acceptable” when used in the context of a carrier, diluent or excipient, refers to a substance that is compatible with the other ingredients of the formulation and is not deleterious to the recipient thereof.

The term “administration of a composition” or “administering a composition” is defined to include the act of providing a compound or pharmaceutical composition of the invention to the subject in need of treatment.

The terms “oral administration” and “orally administering” are broadly defined as a route of administration where a medication is taken through the mouth including “sublingual administration” and “buccal administration” where the medication is placed under the tongue or between the gums and the cheek, respectively, to be absorbed by the body, or to be administered sublingually or buccally as a liquid.

### B. Embodiments of the Invention

As mentioned above, there are provided pharmaceutical compositions that include a therapeutically effective quantity of at least one antiemetic medicament and at least one α-2-adrenergic receptor agonist.

One particular antiemetic that can be used in pharmaceutical compositions described and claimed herein is ondansetron. Other specific, non-limiting examples of antiemetics that can be used include dolasetron, granisetron, palonosetron, promethazine, imenhydrinate, and/or meclizine. All these compounds are described in Table 1 below, which discloses chemical names of such compounds as well as some trade names under which they are also known. Those having ordinary skill in the art can select alternative suitable antiemetics for using in the compositions, if so desired.

TABLE 1
Examples of Antiemetics That Can Be Used in Compositions

| Compound | Chemical Name (IUPAC) | Trade Name(s) |
|---|---|---|
| Ondansetron | (RS)-9-methyl-3-[(2-methyl-1H-imidazol-1- | ZOFRAN ®, ONDISOLV ® |
| | yl)methyl]-2,3-dihydro-1H-carbazol-4(9H)-one | |
| Dolasetron | (2α,6α,8α,9αβ)-octahydro-3-oxo-2,6-methano-2H- | ANZEMET ® |
| | quinolizin-8-yl-1H-indole-3-carboxylate | |
| | monomethanesulfonate, monohydrate | |
| Granisetron | 1-methyl-N-((1R,3r,5S)-9-methyl-9- | KYTRIL ® |
| | azabicyclo[3.3.1]nonan-3-yl)-1H-indazole-3- | |
| | carboxamide | |
| Palonosetron | (3aS)-2-[(3 S)-1-azabicyclo[2.2.2]oct-3-yl]- | ALOXI ® |
| | 2,3,3a,4,5,6-hexahydro-1H-benz[de]isoquinolin-1- | |
| | one | |
| Promethazine | (RS)-N,N-dimethyl-1-(10H-phenothiazin-10- | PHENERGAN ® |
| | yl)propan-2-amine | |
| Dimenhydrinate | 2-benzhydryloxy-N,N-dimethylethanamine; 8- | DRAMAMINE ®, GRAVOL ®, |
| | chloro-1,3-dimethyl-7H-purine-2,6-dione | VOMEX ® |
| Meclizine | (RS)-1-[(4-chlorophenyl)(phenyl)methyl]-4-(3- | BONINE ®, |
| | methylbenzyl)piperazine | BONAMINE ®, ANTIVERT ® |

Those having ordinary skill in the art will select the most appropriate concentration of antiemetic(s) in the pharmaceutical compositions. In determining the proper concentration, those having ordinary skill in the art may choose to select a concentration that may be between about 0.005 and about 0.1 mass %, for example, between about 0.008 and about 0.08 mass %, preferably about 0.01 mass %.

One specific α-2-adrenergic receptor agonist that can be used in pharmaceutical compositions described and claimed herein is dexmedetomidine hydrochloride. Other specific, non-limiting examples of α-2-adrenergic receptor agonists that can be used instead of, or in combination with, dexmedetomidine hydrochloride, include clonidine, guanfacine, xylazine, guanabenz, guanethidine, tizanidine, medetomidine, methylnorepinephrine, norepinephrine, 3-nitrobiphenyline, amitraz, detomidine, and/or lofexidine.

Each of these is also known under one or several trade names as shown in Table 2, below, which also discloses chemical names of such compounds. Those having ordinary skill in the art can select alternative suitable α-2-adrenergic receptor agonists for using in the compositions, if so desired.

TABLE 2
Examples of α-2-Adrenergic Receptor Agonists That Can Be Used in Compositions

| Compound | Chemical Name (IUPAC) | Trade Name(s) |
|---|---|---|
| Dexmedetomidine | 5-[1-(2,3-dimethylphenyl)ethyl]-1H- | PRECEDEX ®, DEXDOR ®, |
| hydrochloride | imidazole hydrochloride | DEXDOMITOR ®, SILEO ® |
| Clonidine | N-(2,6-dichlorophenyl)-4,5-1H- | CATAPRES ®, KAPVAY ®, |
| | imidazol-2-amine | NEXICLON ® |
| Guanfacine | N-(diaminomethylidene)-2-(2,6- | ESTULIC ®, INTUNIV ®, |
| | dichlorophenyl)acetamide | TENEX ® |
| Xylazine | N-(2,6-dimethylphenyl)-5,6-dihydro- | ROMPUN ®, ANASED ®, |
| | 4H-1,3-thiazin-2-amine | SEDAZINE ®, CHANAZINE ® |
| Guanabenz | 2-(2,6-dichlorobenzylidene)hydrazine | WYTENSIN ® |
| | carboximidamide | |
| Guanethidine | 2-[2-(azocan-1-yl)ethyl]guanidine | ISMELIN ®, ESIMIL ® |
| Tizanidine | 5-chloro-N-(4,5-dihydro-1H-imidazol- | ZANAFLEX ®, SIRDALUD ® |
| | 2-yl)benzo[c] [1,2,5]thiadiazol-4- | |
| | amine | |
| Medetomidine | 4-[1-(2,3-dimethylphenyl)ethyl]-3H- | DOMITOR ® |
| | imidazole | |
| Methylnorepinephrine | 4-[(1R,2S)-2-amino-1- | CORBADRINE ® |
| | hydroxypropyl]benzene-1,2-diol | |
| Norepinephrine | 4-(2-amino-1-hydroxyethyl)benzene- | LEVARTERENOL ®, |
| | 1,2-diol | LEVOPHED ®, NOREPIN ® |
| 3-nitrobiphenyline | 2-[1-(3′-nitrobiphenyl-2-yloxy)ethyl]- | N/A |
| | 4,5-dihydro-1H-imidazole | |
| Amitraz | N,N′-[(methylimino)dimethylidyne]- | MITABAN ®, PREVENTIC ®, |
| | di-2,4-xylidine | CERTIFECT ® |
| Detomidine | 4-[(2,3-dimethylphenyl)methyl]-3H- | DORMOSEDAN ® |
| | imidazole | |
| Lofexidine | 2-[1-(2,6-dichlorophenoxy)ethyl]-4,5- | BRITLOFEX ®, |
| | dihydro-1H-imidazole | LUCEMYRA ® |

Those having ordinary skill in the art will select the most appropriate concentration of α-2-adrenergic receptor agonist(s) in the pharmaceutical compositions. In non-limiting, exemplary embodiments, the concentration that may be between about 0.005 and about 0.1 mass %, for example, between about 0.008 and about 0.08 mass %, preferably about 0.01 mass %.

As mentioned above, the compositions may further optionally include an additional component comprising one or several pharmaceutically active compound(s). In such embodiments, the compound(s) that may be used in such additional component are β-blocker(s), or pharmaceutically acceptable salts, hydrates, solvates or N-oxides thereof, as well as pain reliever(s), non-steroid anti-inflammatory drug(s) (NSAIDs), and/or antihistamine medicament(s), all as defined hereinabove.

One particular β-blocker that can be used is metoprolol. Other specific, non-limiting examples of β-blockers or pain relievers that can be used include, propranolol, acebutolol, nadolol, atenolol, betaxolol, esmolol, bisoprolol fumarate, carvedilol, nebivolol, penbutolol, timolol, sotalol, and acetaminophen. Each of these is also known under one or several trade names as shown in Table 3, which also discloses chemical names of such compounds. Those having ordinary skill in the art can select alternative suitable β-blockers for using in the compositions, if so desired.

TABLE 3
Examples of β-Blockers That Can Be Used in Compositions

| Compound | Chemical Name (IUPAC) | Trade Name(s) |
|---|---|---|
| Metoprolol | 1-(isopropylamino)-3-[4-(2-methoxyethyl)phenoxy]propan-2-ol | LOPRESSOR ®, |
| | | TOPROL ® |
| Propranolol | 1-(1-methylethylamino)-3-(1-naphthyloxy)propan-2-ol | CIPLA ®, |
| | | INDERAL ® and |
| | | many others |
| Acebutolol | N-{3-acetyl-4-[2-hydroxy-3-(propan-2- | SECTRAL ®, |
| | ylamino)propoxy]phenyl}butanamide | PRENT ® |
| Nadolol | 5-{[3-(tert-butylamino)-2-hydroxypropyl]oxy}-1,2,3,4- | CORGARD ® |
| | tetrahydronaphthalene-2,3-diol | |
| Atenolol | 2-{4-[2-hydroxy-3-(propan-2- | TENORMIN ® |
| | ylamino)propoxy]phenyl}acetamide | |
| Betaxolol | 1-{4-[2-(cyclopropylmethoxy)ethyl]-phenoxy}-3- | KERLONE ®, |
| | (isopropylamino)propan-2-ol | BETOPTIC ®, and |
| | | others |
| Esmolol | 3-{4-[2-hydroxy-3-(propan-2- | BREVIBLOC ® |
| | ylamino)propoxy]phenyl}propanoate | |
| Bisoprolol | 1-[4-[[2-(1-methylethoxy)ethoxy]methyl]phenoxy]-3[(1- | ZEBETA ® |
| fumarate | methylethyl)amino]-2-propanol-2-butenedioate | |
| Carvedilol | 3-(9H-carbazol-4-yloxy)-2-hydroxypropyl-2-(2- | COREG ®, |
| | methoxyphenoxy)ethylamine | CARVIL ® and |
| | | many others |
| Nebivolol | 2,2′-azanediylbis(1-(6-fluorochroman-2-yl)ethanol) | NEBILET ®, |
| | | BYSTOLIC ® |
| Penbutolol | 1-(tert-butylamino)-3-(2-cyclopentylphenoxy)propan-2-ol | LEVATOL ®, |
| | | LEVATOLOL ® |
| | | and many others |
| Timolol | 1-(tert-butylamino)-3-[(4-morpholin-4-yl-1,2,5-thiadiazol-3- | TIMOPTIC ®, |
| | yl)oxy]propan-2-ol | BETIMOL ® and |
| | | many others |
| Sotalol | N-{4-[1-hydroxy-2-(propan-2- | BETAPACE ® and |
| | ylamino)ethyl]phenyl}methanesulfonamide | others |

As mentioned above, in yet another aspect, the additional component may include one or several non-steroid anti-inflammatory drug(s). In various embodiments, NSAID(s) may be so used in addition to, or instead of, β-blocker(s). Thus, any commonly used NSAID or a combination of several NSAIDs, may be used, e.g., bromfenac, ketorolac, etodolac, sulindac, diclofenac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, or licofelone.

In various embodiments, the additional component may also include an antihistamine medicament, as mentioned above. Non-limiting examples of specific antihistamine medicaments that can be so used include hydroxyzine pamoate, hydroxyzine hydrochloride, diphenhydramine hydrochloride, meclizine, chlorpheniramine, clemastine, promethazine, prochlorperazine, or any combination thereof. Again, antihistamine medicaments may be used in addition to, or instead of, any of the above-mentioned compounds that may be used in the additional component of the composition.

The therapeutically effective quantity of the pharmaceutically active compound(s) comprising the additional component in the entire pharmaceutical composition can be between about 0.1 mass % and about 5.0 mass % of the composition. In various embodiments, the therapeutically effective amount of the third pharmaceutically active compound(s) can be between about 1.0 mass % and about 4.0 mass %, for example, between about 1.5 mass % and 3.0 mass %, preferably about 2.5 mass % of the composition.

Therefore, in various embodiments, the combined quantities of all the pharmaceutically active compounds (i.e., the antiemetic(s), the α-2-adrenergic receptor agonist(s), and the additional compound(s), if used, taken all together) in the entire pharmaceutical composition can be between about 1.3 mass % and about 20.0 mass % of the composition, such as between about 3.0 mass % and about 12.0 mass %, for example, between about 5.0 mass % and about 10.5 mass %, preferably about 10.0 mass % of the composition. Those having ordinary skill in the art will determine the most appropriate quantities of each of the pharmaceutically active compounds that are within the above-mentioned ranges and are most suitable for a particular patient.

The pharmaceutical compositions described herein may contain not only pharmaceutically active components but also, in some embodiments, may further include one or several inactive, neutral compounds which can be pharmaceutically acceptable excipient(s) or carrier(s), including, but not limited to, binder(s), antioxidant(s), adjuvant(s), synergist(s) and/or preservative(s). The mass concentration of such inactive compounds can be between about 80 mass % and about 99 mass % of the entire pharmaceutical composition, such as between about 85 mass % and about 95 mass %, e.g., about 90 mass %.

In various embodiments, the pharmaceutical compositions are formulated as liquid items intended for delivery by injection or some other methods discussed below in more detail. In various embodiments, the pharmaceutical compositions are formulated as solid articles suitable for sublingual or oral administration, such as troches, lozenges, capsules, pills, caps or boluses. These solid compositions typically comprise binder(s) and/or excipient(s). They can be prepared by first mixing the pharmaceutically active compounds described above with suitable binder(s) and/or excipient(s) followed by molding or compressing the blend. Both hard and chewable lozenges and troches are within the scope of the invention.

Typical binder(s) that can be used for fabricating the solid articles mentioned above include, without limitation, polyglycols as defined above, such as, e.g., polyethylene glycols (PEGs), polyethylene oxides (POE), methoxypolyethylene glycols, polypropylene glycols, polybutylene glycols or derivatives thereof having a molecular weight that is sufficient to provide the necessary hardness and time for dissolution of the troche; for example, the acceptable molecular weight can be within the range of between about 1,000 Daltons and about 8,000 Daltons. In some embodiments PEG-1450 or PEG-400 can be used. Non-limiting examples of some specific polyglycol derivatives that can be used are:

(a) PEG-laureates and dilaureates (e.g., PEG-10-, PEG-12-, PEG-20-, PEG-32-laurates, PEG-20- and PEG-32-dilaurates, PEG-20-glyceryl-, PEG-30-glyceryl- and PEG-40-glyceryl-laurates, PEG-80-sorbitan laurate);

(b) PEG-oleates, dioleates and trioleates (e.g., PEG-12-, PEG-15-, PEG-20-, PEG-32, PEG-200- and PEG-400-oleates, PEG-20- and PEG-32-dioleates, PEG-20-trioleate, PEG-25-glyceryl trioleate, PEG-20-glyceryl- and PEG-30-glyceryl-oleates, PEG-40-sorbitan oleate);

(c) PEG-stearates and distearates (e.g., PEG-15-, PEG-40-, PEG-100-stearates, PEG-32-distearate and PEG-20-glyceryl stearate);

(d) castor, palm kernel, corn and soya oil derivatives of PEG (e.g., PEG-35-, PEG-40-and PEG-60-castor oils, PEG-40-, PEG-50- and PEG-60-hydrogenated castor oils, PEG-40-palm kernel oil, PEG-60-corn oil, PEG-30-soya sterol);

(e) other PEG derivatives (e.g., PEG-24- and PEG-30-cholesterol, PEG-25-phytosterol, PEG-6- and PEG-8-caprate/caprylate glycerides, tocopheryl PEG-100 succinate, PEG-15-100 octylphenol products and PEG-10-100 nonylphenol products); and

(f) other products such as polyglyceryl-10-laurate, POE-9- and POE-23-lauryl ethers, POE-10- and POE-20-oleyl ethers, POE-20-stearyl ether, polysorbates-20 and 80, polyglyceryl-10-oleate, Tween 40, Tween 60, sucrose monostearate, monolaurate and monopalmitate and various products of Poloxamer series.

Typical excipient(s) that can be used for fabricating the solid articles mentioned above include, without limitation, gelatin, sodium saccharin, stevioside, peppermint oil, or any natural or artificial fruit, vegetable, flower, beverage or candy flavor.

As stated above, the compositions may optionally further comprise one or several antioxidant(s). If antioxidant(s) are used, non-limiting examples of those that can be used include α-tocopherol acetate, acetone sodium bisulfite, acetylcysteine, ascorbic acid, ascorbyl palmitate, butylated hydroxyanisole, butylated hydroxytoluene, cysteine, cysteine hydrochloride, tocopherol natural, tocopherol synthetic, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea and tocopherols.

As stated above, the compositions may optionally further include one or several adjuvant(s) or synergist(s). If adjuvant(s) or synergist(s) are used, non-limiting examples of those that can be used include citric acid, EDTA (ethylenediaminetetraacetate) and salts, hydroxyquinoline sulfate, phosphoric acid and tartaric acid.

As stated above, the compositions may optionally further include one or several preservative(s). If preservative(s) are used, non-limiting examples of those that can be used include benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sorbic acids and salts, β-phenylethyl alcohol and thimerosal.

In various embodiments, the pharmaceutical compositions described herein can be administered to a subject in need of conscious sedation, procedural sedation, analgesia and/or pre-sedation, and in general, for any kind of non-general anesthesia, by various local administrations. More specifically, the pharmaceutical formulations described herein may be prescribed by ordinarily skilled medical practitioners such as physicians, as a means of conscious sedation or pre-sedation. This is intended to be used for certain patients who experience or expect to experience high anxiety, bouts of panic attacks, disquietude, apprehension, angst or similar feelings of psychological discomfort or distress prior to, or during, medical or surgical procedures as described in more detail below. The patients may be of any age, including children, adolescents and adults.

For example, the formulation can be used prior to various outpatient surgeries and medical procedures, both invasive and non-invasive, such as an ophthalmic surgery, outpatient medical or surgical procedures, dental procedures, urological procedures, obstetric and gynecological procedures, gastrointestinal procedures, otolaryngological procedures, cosmetic surgery procedures, dermatological procedures, podiatric procedures, orthopedic procedures, emergency medical treatments, psychiatric treatments, and veterinarian procedures.

Specific representative examples of the procedures that are amenable to use of the formulation include, without limitation, cataract surgery, glaucoma surgery, corneal surgery, eyelid surgery, retinal surgery, tooth extraction, oral surgery, root canal surgery, medical imaging procedures (e.g., Mill or CAT scanning, especially for patients suffering from claustrophobia), biopsy, bone marrow harvesting, colonoscopy, endoscopy and laparoscopy.

In various embodiments, the local administration is by oral route, such as sublingually or buccally, typically being delivered to the patient in the form of a solid delivery vehicle such as a troche, a lozenge, a capsule, a pill, a cap, and a bolus, as mentioned above. However, in various embodiments, the pharmaceutical composition may be formulated as a liquid item adapted for sublingual or buccal administration (in which case it will include all the pharmaceutically active compounds described above, but no pharmaceutically suitable binder); such liquid formulations may be delivered by any method to be selected by one having ordinary skill in the art of delivery of medications, e.g., via a syringe or a pipette. Other exemplary methods of local administration that may be used instead of intravenous administration or to complement the latter, as appropriate, include without limitation rectal administration, nasal spray administration, oral spray administration, transdermal administration, and transmucosal administration.

It will be understood by those having ordinary skill in the art that the specific dose level and frequency of dosage for any particular patient may be varied and will depend upon many factors including the activity of the specific compound employed, the metabolic stability and length of action of that compound, the age, body weight, general health, gender, diet and the severity of the particular condition being treated.

In additional embodiments, the pharmaceutical compositions of the present invention may be incorporated into vehicles allowing extended release of the compositions over a period of time. To achieve this effect, the compositions may be combined with polymers forming such vehicles. The product will therefore be an extended release capsule, gel tablet, liquid table, or the like, ensconcing the pharmaceutical formulation, or alternatively a matrix polymer structure holding the pharmaceutical formulation that is embedded into the matrix.

The vehicle carrying the pharmaceutical formulation may be configured to allow the gradual release of the pharmaceutical formulation over not less than about 15 minutes, not less than about 1 hour, or not less than about 12 hours, e.g., between about 15 minutes and about 20 hours, such as between about 12 hours and about 20 hours, for example, about 16 hours. The rate of release may be uniform throughout the entire period of release; alternatively, those having ordinary skill in the art may formulate the release vehicle in such a way as to allow different rates of release at different times, for example, faster release at the beginning of the process of release and slower at later stages, or vice versa, or in any other way that may be necessary.

The vehicle may be manufactured from any pharmaceutically acceptable polymer that is capable of releasing at least 95 mass % of the pharmaceutical formulation that the vehicle incorporates within the above-mentioned time periods, i.e., within not less that about 15 minutes, not less than about 1 hour, or not less than about 12 hours, e.g., between about 15 minutes and about 20 hours, such as between about 12 hours and about 20 hours, for example, about 16 hours. In various embodiments, the vehicle may be formulated to ensure the release of at least 97 mass % of the pharmaceutical formulation, for example, at least 99.5 mass %.

Those having ordinary skill in the art will select the most appropriate polymer for making the vehicle. As guidance only, some non-limiting examples of such polymers include esters of cellulose, e.g., methyl cellulose and hydroxypropyl methyl cellulose. Other acceptable polymers are poly(lactic-co-glycolic acid) (PLGA), polylactic acid, polyglycolide, dextrin, polyacetals, poly(N-(2-hydroxypropyl)methacrylamide), polycaprolactone, and poly-3-hydroxybutyrate.

One particular type of product that can be used in fabricating the vehicle carrying the pharmaceutical formulation may be water-soluble methylcellulose and hydroxypropyl methylcellulose polymers, such as METHOCEL® family of products, for example, a hydroxypropyl methylcellulose product METHOCEL® E4M, 20% METHOCEL®K4M, or 10% METHOCEL® K100 or, alternatively and particularly useful for hot melt extrusion, another hydroxypropyl methylcellulose product, AFFINISOL™HPMC (all mentioned hydroxypropyl methylcellulose polymers are available Dow Chemical Co. of Midland, Mich.).

While all the products that include vehicles carrying the pharmaceutical compositions of the present invention are useful for inducing sedation prior to or during all the medical, surgical and other procedures mentioned above, those having ordinary skill in the art may find these systems particularly suitable and advantageous in the treatments of depression (including major depression or treatment-resistant depression), PTSD, alcohol or substance abuse/dependence, suicide prevention, anxiety (including generalized anxiety disorder), personality disorders (inclusive of borderline personality disorder), and related psychiatric and/or psychological disorders, syndromes, symptoms, maladies, and the like. Thus, the present invention contemplates methods of treating such disorders, syndromes, symptoms, and/or maladies by administering the pharmaceutical compositions described herein.

In another aspect, methods for fabricating the above-described pharmaceutical compositions are provided. In various embodiments, a one-batch formulation method may be used, where the components of the pharmaceutical formulation are combined in a single container; the components may be added to the container simultaneously or consecutively. Alternatively, a two- or multiple-batch method(s) may be used if desired, where each component of the pharmaceutical formulation can be combined in a separate container followed by combining the contents of each container.

In one exemplary, non-limiting procedure, pre-measured quantities of each ingredient in the form of dry powders can be mixed to form a dry blend followed by mixing the blend with a pre-molten troche base. The composition can then be poured into a mold or molded to form a troche.

In another aspect, pharmaceutical kits are provided. In various embodiments, the kit includes a sealed container approved for the storage of solid pharmaceutical compositions, the container containing one of the above-described pharmaceutical compositions, an instruction for the use of the composition, and information about the composition, which may be affixed to the container or otherwise enclosed with it.

The following examples are provided to further elucidate the advantages and features of the present invention but are not intended to limit the scope of the invention. The examples are for the illustrative purposes only. USP pharmaceutical grade products were used in preparing the formulations described below.

### Example 1. Preparing a Pharmaceutical Composition #1

A pharmaceutical composition may be prepared as described below. The following products can be used in the amounts and concentrations specified:
(a) about 0.01 g of dexmedetomidine hydrochloride;
(b) about 0.2 g of ondansetron;
(c) about 0.2 g of ondansetron hydrochloride;
(d) about 1 mL of lemon oil flavoring; and
(e) about 15.5 g of standard troche base (comprising polyglycol 1450, polyglycol 400, gelatin, sodium saccharin and steviaside).

The troche base can be melted at low heat while being stirred; when completely molten, the heat can be turned off with continued stirring. All the pre-weighed dry ingredients can be added into the molten base followed by addition of the flavoring, and mixing all components together.

While any shape may be used, a half-moon shaped troche mold can be lightly sprayed with PAM® (or a suitable oil/releasing agent) to cover the entire surface of the mold. The mixture prepared as explained above can then be poured into the mold and allowed to cool and harden at room temperature. A heat gun can then be used to smooth out the surface followed by another round of cooling at room temperature. Once cooled, the so prepared troche may be removed from the mold and placed into a labeled prescription vial. The troche is now ready to be administered.

### Example 2. Preparing a Pharmaceutical Composition #2

Another pharmaceutical composition may be prepared using the same components as for pharmaceutical composition #1 in Example 1, above, with the addition of about 0.2 g of propranolol hydrochloride. The composition can be then shaped in the form of a troche in the same fashion described in Example 1, above.

Although the invention has been described with reference to the above examples, it will be understood that modifications and variations are encompassed within the spirit and scope of the invention. Accordingly, the invention is limited only by the following claims.

What is claimed is:

1. A pharmaceutical composition, comprising a therapeutically effective quantity of a pharmaceutical formulation incorporated into a vehicle, wherein the pharmaceutical composition comprises:
(a) a therapeutically effective quantity of an at least one antiemetic medicament;
(b) a therapeutically effective quantity of an at least one α-2-adrenergic receptor agonist; and
(c) optionally, a pharmaceutically acceptable excipient,
wherein the vehicle further optionally provides for extended release of the pharmaceutical formulation.

2. The pharmaceutical composition of claim 1, wherein the antiemetic medicament is selected from the group consisting of ondansetron, dolasetron, granisetron, palonosetron, promethazine, imenhydrinate, and meclizine.

3. The pharmaceutical composition of claim 2, wherein the antiemetic medicament is ondansetron.

4. The pharmaceutical composition of claim 1, wherein the α-2-adrenergic receptor agonist is selected from the group consisting of dexmedetomidine hydrochloride, clonidine, guanfacine, xylazine, guanabenz, guanethidine, tizanidine, medetomidine, methylnorepinephrine, norepinephrine, 3-nitrobiphenyline, amitraz, detomidine, and lofexidine.

5. The pharmaceutical composition of claim 4, wherein the α-2-adrenergic receptor agonist is dexmedetomidine hydrochloride.

6. The pharmaceutical composition of claim 1, wherein the excipient is selected from the group consisting of extended release capsules ensconcing the pharmaceutical formulation and a matrix polymer structure holding the pharmaceutical formulation that is embedded into the matrix.

7. The pharmaceutical composition of claim 6, wherein the excipient is configured to allow the release of the pharmaceutical formulation in a period of time of between about 15 minutes and about 20 hours.

8. The pharmaceutical composition of claim 6, wherein the excipient comprises a polymer selected from the group consisting of esters of cellulose, poly(lactic-co-glycolic acid), polylactic acid, polyglycolide, dextrin, polyacetals, poly(N-(2-hydroxypropyl)methacrylamide), polycaprolactone, and poly-3-hydroxybutyrate.

9. The pharmaceutical composition of claim 8, wherein the esters of cellulose are selected from the group consisting of methyl cellulose and hydroxypropyl methyl cellulose.

10. The pharmaceutical composition of claim 6, wherein the excipient comprises a substance selected from the group consisting of gelatin, sodium saccharin, stevioside, peppermint oil, cherry flavor, lemon oil, raspberry flavor and combinations thereof.

11. The pharmaceutical composition of claim 1, wherein the pharmaceutical formulation further comprises a therapeutically effective quantity of one or more additional pharmaceutically active compounds selected from the group consisting of β-blockers, NSAIDs, antihistamines, pain relievers and combinations thereof, or pharmaceutically acceptable salts, hydrates, solvates or N-oxides thereof.

12. The pharmaceutical composition of claim 11, wherein the β-blocker or the pain reliever is selected from the group consisting of metoprolol, propranolol, acebutolol, nadolol, atenolol, betaxolol, esmolol, bi soprolol fumarate, carvedilol, nebivolol, penbutolol, timolol, sotalol, and acetaminophen.

13. The pharmaceutical composition of claim 11, wherein the NSAID is selected from the group consisting of bromfenac, ketorolac, etodolac, sulindac, diclofenac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, and licofelone.

14. The pharmaceutical composition of claim 11, wherein the antihistamine is selected from the group consisting of hydroxyzine pamoate, hydroxyzine hydrochloride, diphenhydramine hydrochloride, meclizine, chlorpheniramine, clemastine, promethazine, and prochlorperazine.

15. A method for inducing conscious sedation, procedural sedation, analgesia, pre-sedation or non-general anesthesia in a patient comprising administering to a patient in need thereof the pharmaceutical composition of claim 1 prior to carrying out an invasive or a non-invasive medical procedure.

16. The method of claim 15, wherein the invasive or non-invasive medical procedure is selected from the group consisting of an ophthalmic surgery, a dental procedure, an outpatient medical procedure, an obstetric and gynecological procedure, a gastrointestinal procedure, an otolaryngological procedure, a cosmetic surgery procedure, a dermatological procedure, a podiatric procedure, an orthopedic procedure, an emergency medical treatment, a psychiatric treatment, a urological procedure, and a veterinarian procedure.

17. The method of claim 16, wherein the ophthalmic surgery is selected from the group consisting of a cataract surgery, a glaucoma surgery, corneal surgery, eyelid surgery, and retinal surgery.

18. The method of claim 16, wherein the dental procedure is selected from the group consisting of a tooth extraction, an oral surgery, and a root canal surgery.

19. The method of claim 16, wherein the outpatient surgical procedure is selected from the group consisting of a medical imaging procedure, biopsy, bone marrow harvesting, colonoscopy, endoscopy, and a laparoscopic procedure.

20. The method of claim 16, wherein the patient experiences or expects to experience high anxiety, bouts of panic attacks, disquietude, apprehension, or angst prior to, or during, the medical procedure.

21. The method of claim 16, wherein the pharmaceutical composition is administered by a method selected from the group consisting of sublingual administration, buccal administration, rectal administration, nasal spray administration, oral spray administration, transdermal administration, and transmucosal administration.
